(19) United States
(12) Patent Application Publication
(10) Pub. No.: US 2019/0105467 A1
(43) Pub. Date: Apr. 11, 2019

(54) Modified Tip for a Sheath Assembly

(71) Applicant: Baylis Medical Company Inc., Montreal (CA)

(72) Inventors: Ros Thomas Brannick, East York (CA); Brian Ma, Toronto (CA); Audrey Man Yin Chan, Richmond Hill (CA); Melanie Thompson Smith, Toronto (CA); Alice Zhang, Toronto (CA); Jan-Hung Chen, Georgetown (CA)

(21) Appl. No.: 16/157,783

(22) Filed: Oct. 11, 2018

Related U.S. Application Data
(60) Provisional application No. 62/571,019, filed on Oct. 11, 2017.

(51) Int. Cl. A61M 25/01; A61M 25/00; A61M 39/02

(52) U.S. Cl. CPC A61M 25/0108; A61M 25/0068; A61M 2039/0279; A61M 39/0247; A61M 25/008

(57) ABSTRACT

A method and apparatus are disclosed for a modified tip for a sheath assembly that provides enhanced radiopacity while maintaining the distal tip profile to maintain adequate tip stiffness and tip transition force. The disclosed method and apparatus provide a distal tip segment for a sheath assembly comprising a region of increased wall thickness along a portion or section of the distal tip segment, providing increased cross-sectional area there-along to provide enhanced radiopacity, and a distal most section of the distal tip segment having a reduced or minimal profile to provide an atraumatic tip while maintaining sufficient stiffness for crossability. The distal tip segment is operable to be coupled to a proximal shaft portion of the sheath assembly, to provide enhanced radiopacity while maintaining the distal tip profile to maintain adequate tip stiffness.

### TECHNICAL FIELD

The disclosure relates to a modified tip for a sheath assembly. More specifically, the present disclosure relates to a modified tip that provides enhanced radiopacity while maintaining the distal tip profile to maintain adequate tip stiffness and tip transition force.

### BRIEF DESCRIPTION OF THE DRAWINGS

In order that the invention may be readily understood, embodiments of the invention are illustrated by way of examples in the accompanying drawings, in which:

FIG. 1A-1E are an illustration of a modified tip for sheath assembly in accordance with various embodiments of the present invention; and

FIGS. 2A-2B are an illustration of a modified tip for a sheath assembly in accordance with various embodiments of the present invention.

### DETAILED DESCRIPTION

Some medical procedures may require the use of a sheath (such as a fixed curve sheath or a steerable sheath) in order to reach a desired location within a patient's body in order to treat the patient. The sheath may provide a means (such as a marker or an indicator) for the user to see one or more regions of the sheath under imaging such as fluoroscopy. However, the means may not be sufficient to provide an indication of the entire tip profile. Some prior art systems provide sheaths that comprise one or more polymer segments of enhanced radiopacity. The polymer segments of enhanced radiopacity may be provided with a radiopaque material such as barium. Even when the polymer segment having enhanced radiopacity is provided with a very high or maximum amount of available radiopacity [such as barium 40%], the amount of radiopacity may not be sufficient to enable visibility of the tip and may not provide a sharp contrast to define a boundary of the tip.

The inventors of the present invention have discovered a unique problem associated with prior art sheath assemblies that do not provide sufficient radiopacity and as such are not able to provide sharp contrast at the distal tip region. Due to insufficient radiopacity and lack of contrast the physician may not be able to ascertain where the exact location of (a specific region of) the sheath, such as where the distal tip is located.

The inventors of the present invention have additionally discovered a unique solution that attempts to solve the above problem by increasing the wall thickness of the radiopaque polymer at the distal tip region.

The inventors of the present invention have additionally discovered a unique problem associated with increasing the wall thickness in the distal tip region in order to increase tip stiffness and increase the amount of force required to cross or the tip transition force (for example in a transseptal procedure) which may decrease cross-ability of the sheath.

As will be presently described, the present inventors have additionally discovered a unique solution to solving the problem by providing a means for increasing radiopacity at the tip while maintaining the distal tip profile to maintain adequate tip stiffness and tip transition force. In other words, the present inventors have discovered a unique solution that solves the problem associated with prior art sheath assemblies while still maintaining the desired tip profile and the tip transition force or crossing force.

The present inventors have discovered and invented embodiments for providing sufficiently high radiopacity at the tip, where in some examples the tip radiopacity provides sufficiently high/sharp contrast to allow the physician to ascertain the distal tip profile or the boundary of the distal tip under imaging (such as fluoroscopy), while ensuring that the tip is still soft enough to provide atraumaticity and is sufficiently stiff to provide good cross-ability.

More specifically, some embodiments of the present invention provide increasing the wall thickness along a segment of the distal tip having radiopacity (such as by using a radiopaque polymer) which increases the cross-sectional area of the radiopaque region to allow for enhanced radiopacity, while substantially providing/maintaining a minimal or reduced wall thickness or profile along a distal most segment of the tip which provides an atraumatic tip that is still sufficiently stiff to provide good transition force for crossability.

In one broad aspect, embodiments of the present invention comprise a distal tip segment for a sheath assembly comprising: a region of increased wall thickness along a portion/section of the distal tip segment, the region having an increased cross-sectional area to provide enhanced radiopacity; and a distal most straight section of the distal tip segment having a reduced/minimal profile to provide an atraumatic tip while maintaining sufficient stiffness for crossability, the distal most straight section having a region of reduced wall thickness wherein the distal tip segment is operable for coupling to a proximal shaft portion of the sheath assembly, to provide enhanced radiopacity while maintaining the distal tip profile for adequate tip stiffness.

As a feature of this broad aspect, the distal most section of the distal tip segment provides a reduced/good tip transition force to cross a region of tissue to facilitate a smooth transition.

As another feature of this broad aspect, the distal tip segment additionally comprises a proximal section of enhanced thickness that defines the region of increased wall thickness along a proximal portion of the distal tip segment.

As another feature of this broad aspect, the distal tip segment provides/comprises a gradual transition between the proximal section of enhanced wall thickness and the distal most section of the distal tip segment.

As a further feature of this broad aspect, the distal tip segment provides a gradual transition from the proximal shaft to the distal most section of the distal tip segment.

As still another feature of this broad aspect, the region of increased wall thickness along the distal most section of the distal tip segment has a wall thickness of about 0.010″ to about 0.014″.

As an example of this feature, the region of increased wall thickness along the distal most section of the distal tip segment has a tip wall thickness of about 0.012″.

As a feature of this broad aspect, the region of increased wall thickness along the proximal section of enhanced wall thickness has a maximum outer diameter that is less than about to about 0.158″.

As still another feature of this broad aspect, wherein the distal tip segment has an inner diameter (along the region of increased wall thickness) that is in the range of between about 0.110″ to about 0.112″.

As still another feature of this broad aspect, the distal tip segment has inner diameter along the region of increased wall thickness that is equal to about 0.111″.

As another feature of this broad aspect, the distal tip segment along the proximal section of enhanced wall thickness has a wall thickness that ranges from about 0.010″ at an interface with the distal most section of the distal tip region, to about 0.024″ at an interface with the proximal shaft portion.

In some embodiments of the present invention, the distal tip segment along the region of increased wall thickness has a wall thickness that ranges from about 0.010″ at an interface with the region of reduced wall thickness to about 0.024″ at an interface with the proximal shaft portion.

As a feature of this broad aspect, a lap joint is provided at an interface between the distal tip segment and the proximal shaft portion of the sheath assembly, wherein the lap joint defines the proximal of enhanced wall thickness. As an example of this feature, the lap joint is formed between a polymer layer of the proximal shaft portion and a polymer layer of the distal tip segment.

In one instance of this example, the polymer layer of the proximal shaft portion is rolled down at the interface between the proximal section of enhanced wall thickness, of the distal tip segment, so it is tapered to enable the polymer layer of the distal tip segment to be joined/formed thereon in an overlapping configuration.

In some embodiments, the polymer layer of the proximal shaft portion comprises Nylon 12 and the polymer layer of the distal tip segment including the proximal section of enhanced wall thickness comprises a 35D Pebax. In one such example, the polymer layer of the proximal shaft portion comprises 25% barium and the polymer layer of the region of increased wall thickness of the distal tip segment (including the distal most section of the distal tip region and the proximal section of enhanced wall thickness) comprises 40% barium to provide a region of enhanced radiopacity providing an area/region of sharp contrast. In one instance of this example, the polymer layer of the proximal shaft portion comprises Nylon 12 and the polymer layer of the distal tip segment comprises a 35D Pebax. In some such examples, the polymer segments along the proximal shaft 110 may comprise 25% barium.

In some embodiments, the stiffness of the distal most section 600A of the distal tip region is less than about 0.8 N, [which in some embodiments of the present invention may be defined as the force required to compress the tip by about 1 mm]. In some embodiments, the (average) stiffness of the distal most section of the distal tip region is less than about 0.5 N. In some embodiments, the stiffness of the distal most section of the distal tip region ranges from between about 0.25 N to about 0.65 N. In some embodiments, the stiffness of distal most section of the distal tip region ranges from between about 0.4 N to about 0.6 N.

In some embodiments, the radiopacity of the region of reduced wall thickness along the distal most section of the distal tip segment has a radiopacity of between about 1.6 mmAl (mm of Aluminum) to about 1.9 mmAl. In one example, the radiopacity of the region of reduced wall thickness along the distal most section of the distal tip has a (or an average value of) radiopacity of about 1.8 mmAl.

In some embodiments, the radiopacity of the section of enhanced wall thickness of the distal tip segment along the distal most section just distal of holes has a radiopacity that is greater than a value of between about 1.6 mmAl to about 1.9 mmAl.

As still another feature of this broad aspect, the distal tip segment has tip transition force [which is in some examples a measure of force that is used to cross a region of tissue such as the septum for a transseptal procedure] of less than about 4N to cross a region of tissue. As still another feature of this broad aspect, the tip transition force is between about 0.5N to about 3.0N. In one specific example, the tip transition force is about 1.6N.

In some embodiments of the present invention, the tip transition force defines the force usable to cross the distal tip segment through a region of tissue, where the sheath assembly is usable with a dilator inserted therein/needle.

With specific reference now to the drawings in detail, it is stressed that the particulars shown are by way of example and for purposes of illustrative discussion of certain embodiments of the present invention only. Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

### Distal Tip Architecture

### Region of Increased Wall Thickness

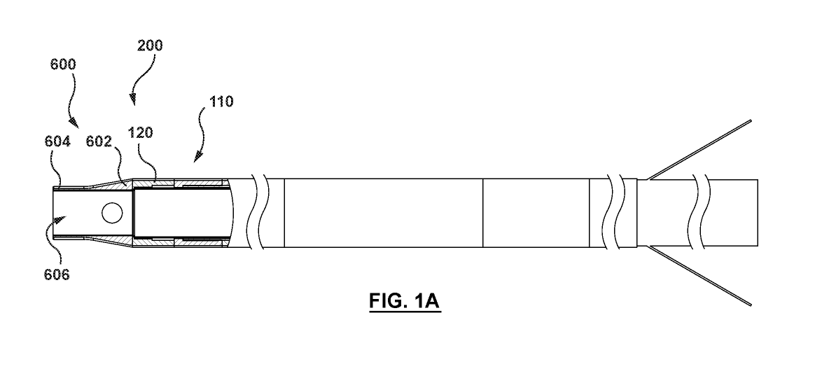

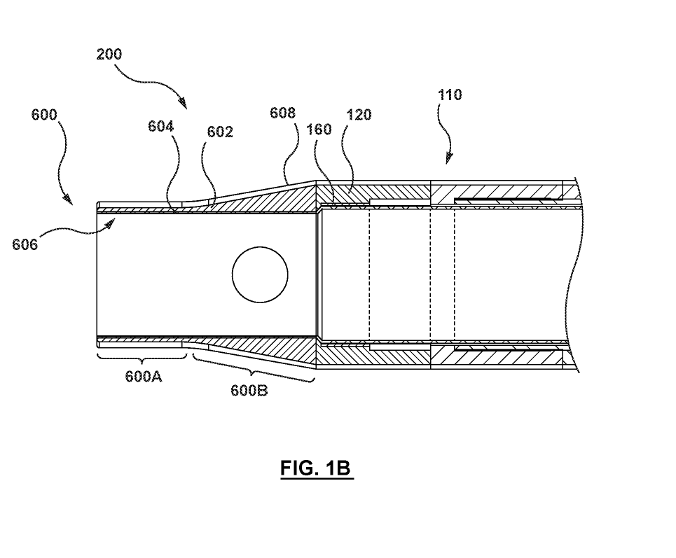

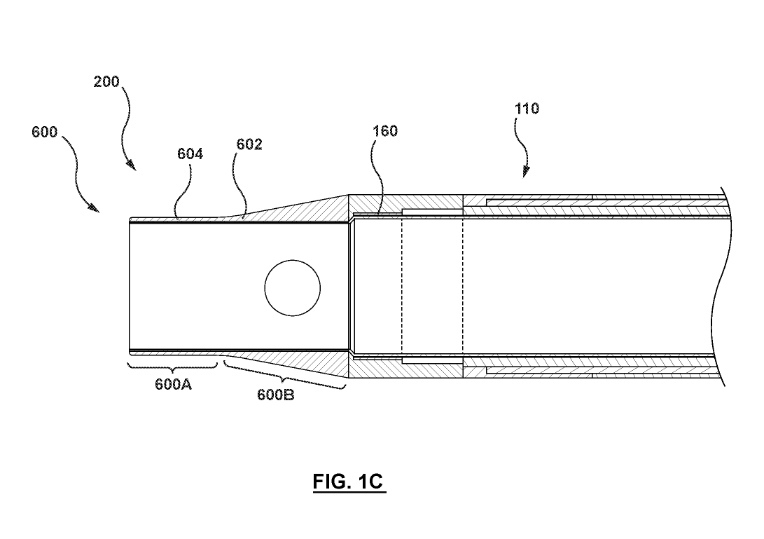

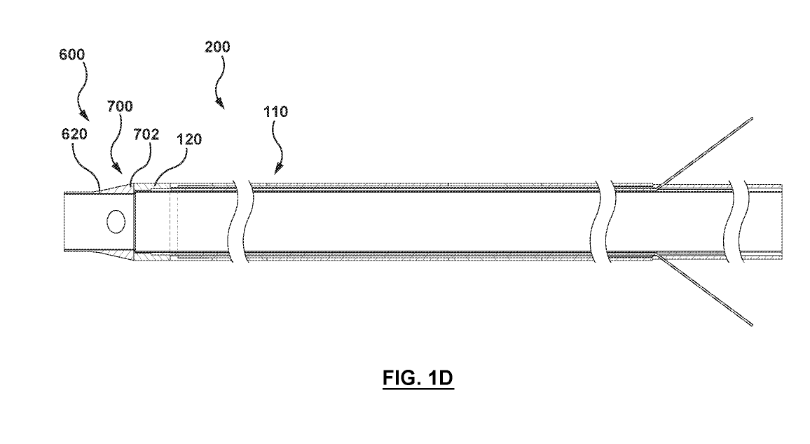

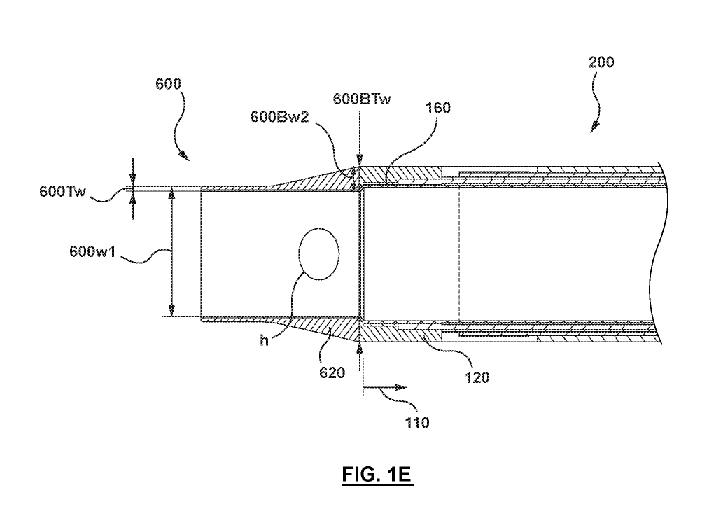

In one embodiment of the present invention, as shown in FIG. 1A and FIG. 1B, a modified tip is provided for a sheath assembly 200. The modified tip 600 provides enhanced radiopacity while maintaining the distal tip profile to maintain adequate tip stiffness and tip transition force. More specifically, a distal tip segment 600 is provided for a sheath assembly 200 that defines a modified tip. The distal tip segment 600 comprises a region of increased wall thickness 606 along a portion/section of the distal tip segment 600. The region of increased wall thickness 606 provides increased cross-sectional area. In some embodiments, the area of increased wall thickness 606 is radiopaque to provide enhanced radiopacity.

### Distal Tip Section

The distal tip segment 600 comprises a distal most section or distal tip section 600A (or in other words a region of reduced wall thickness 600A). In one such example, the distal most section 600A comprises a substantially straight segment. Some such embodiments of the present invention, the distal tip segment 600 has a reduced/minimal profile to provide an atraumatic tip while maintaining sufficient stiffness for crossability. The distal tip segment 600 is operable to be coupled to a proximal shaft portion 110 of the sheath assembly 200, to provide enhanced radiopacity while maintaining the distal tip profile to maintain adequate tip stiffness. In some examples of the present embodiment, as additionally shown in FIG. 1C, the distal most section 600A of the distal tip segment 600 helps to provide a reduced/good tip transition force to cross a region of tissue to facilitate a smooth transition.

### Proximal Section of Enhanced Thickness

### Gradual Transition

The distal tip segment 600 additionally comprises a proximal section of enhanced wall thickness 600B that defines the region of increased wall thickness along a proximal portion of the distal tip segment 600. In some such examples, the distal tip segment 600 provides/comprises a gradual transition between the proximal section of enhanced wall thickness 600B and the distal most section of the distal tip segment 600A. As shown in FIGS. 1B and 1C, in one such example, the proximal section of enhanced wall thickness 600B defines a taper 608. The outer diameter of the taper 608 gradually increases from a narrower distal portion (e.g., at the distal most section 600A) to a wider proximal portion. In the example shown in FIGS. 1A to 1E, the outer diameter of the proximal portion of taper 608 is substantially the same as the diameter of the outer diameter of the distal end of the proximal shaft 110. As such, in some such examples, the distal tip segment 600 provides a gradual transition from a proximal shaft 110 of the sheath assembly 200 to the distal most section 600A of the distal tip segment 600.

### Interface Between Distal Tip Segment and the Proximal Shaft Portion

In some embodiments, of the present invention, as discussed with reference to FIG. 1D, the region 702 comprises a lap joint at an interface 700 between the distal tip segment 600 and the proximal shaft portion 110 of the sheath assembly 200. The lap joint as proximate the proximal surface of enhanced wall thickness 600B. As an example of this feature, the lap joint is formed between a polymer layer 120 of the proximal shaft portion 110 and a polymer layer 620 of the distal tip segment. In an embodiment, the lap joint comprises features of both the polymer layer 120 and polymer layer 620 which overlap and engage in more than one plane (not shown). Other ways of joining the distal tip segment 600 and the proximal shaft portion 110 are contemplated, including, for example, a butt joint.

In one instance of this example, the polymer layer 120 of the proximal shaft portion 110 is rolled down at the interface 700 between the proximal section of enhanced wall thickness 600B, of the distal tip segment 600, such that it is tapered to enable the polymer layer 620 of the distal tip segment 600 to be joined/formed thereon in an overlapping configuration (not shown). In some such examples, a combination of the polymer layer 120 of the proximal shaft portion 110 and the polymer layer 620 of the distal tip segment 600 help define the taper 608 of the proximal section of enhanced wall thickness 600B.

### Tip Wall Thickness

In a specific embodiment of the present invention, the region of increased wall thickness 606 along the distal most section 600A of the distal tip segment 600 has a wall thickness 600Tw that is between about 0.010″ to about 0.014″, as additionally shown in FIG. 1E. In a specific example, the region of increased wall thickness 606 along the distal most section 600A of the distal tip segment 600 has a tip wall thickness 600Tw of about 0.012″.

In a specific embodiment of the present invention, the region of increased wall thickness 606 along the proximal section of enhanced wall thickness 600B has an outer diameter (OD) shown by reference number 600BTw that is less than about 0.158″ (for example, a maximum outer diameter (OD)). In some such examples, the distal tip segment 600 has an inner diameter 600w1, (along the region of increased wall thickness including the distal most section 600A and the proximal section of enhanced thickness 600B) that is in the range of between about 0.110 to about 0.112″. In a specific instance, the distal tip segment 600 has inner diameter 600w1 along the region of increased wall thickness that is equal to about 0.111″. In one specific example, the distal tip segment 600, along the proximal section of enhanced wall thickness 600B has a wall thickness 600Bw2 that ranges from about 0.010″ at an interface with the distal most section 600A of the distal tip region, to about 0.024″ at an interface with the proximal shaft portion 110.

In some such examples, the shaft proximal portion 110 has a shaft inner diameter 0.0118 inches+/−0.001 inches, and an outer diameter of between about 0.0158″ to about 0.160″.

In some such embodiments of the present invention, the wall thickness along the distal tip segment 600 (including along the distal most section 600A and proximal section of enhanced wall thickness 600B) may impact one or more of enhanced radiopacity, tip stiffness and crossing force. In some embodiments of the present invention, wall thickness is provided that provides one or more of optimal tip stiffness (to provide a substantially atraumatic) and smooth tip transition to provide a substantially low tip transition or crossing force for providing a smooth transition, while at the same time increasing or enhancing the radiopacity of the distal tip segment 600 to enable the tip profile to be visible under imaging.

### Radiopacity

In some embodiments, the polymer layer 120 of the proximal shaft portion 110 comprises Nylon 12 and the polymer layer of the distal tip segment 620 including the proximal section of enhanced wall thickness 600B comprises a 35D Pebax. In one such example, the polymer layer 120 of the proximal shaft portion comprises 20% barium and the polymer layer 620 of the region of increased wall thickness of the distal tip segment 600 (including the distal most section 600A of the distal tip region 600 and the proximal section of enhanced wall thickness 600B) comprises 40% barium to provide a region of enhanced radiopacity. The region of enhanced radiopacity thus provides an area/region of sharp contrast to enable the distal tip region 600 to be particularly visible under imaging.

In some such embodiments, the wall thickness of the distal tip segment 600 and the architecture thereof formed by the lap joint 702 formed between radiopaque sections of the proximal shaft 110 and the distal tip segment 600 provide a sharp contrast to define a boundary of the distal tip segment 600, and still provide a tip wall thickness 600Tw that provides an atraumatic tip that is still sufficiently stiff to provide good transition force for crossability. In other words the distal tip segment 600 comprises an architecture enabling enhanced radiopacity by providing a distinct region of sharp contrast or a high contrast area near the tip of the sheath 200 (or at the tip of the distal tip region 600) to give the user a clear indication of where the distal tip of the sheath 200 is positioned during the procedure. At the same time, the architecture provides for a good tip transition force for ease of crossing while also being atraumatic.

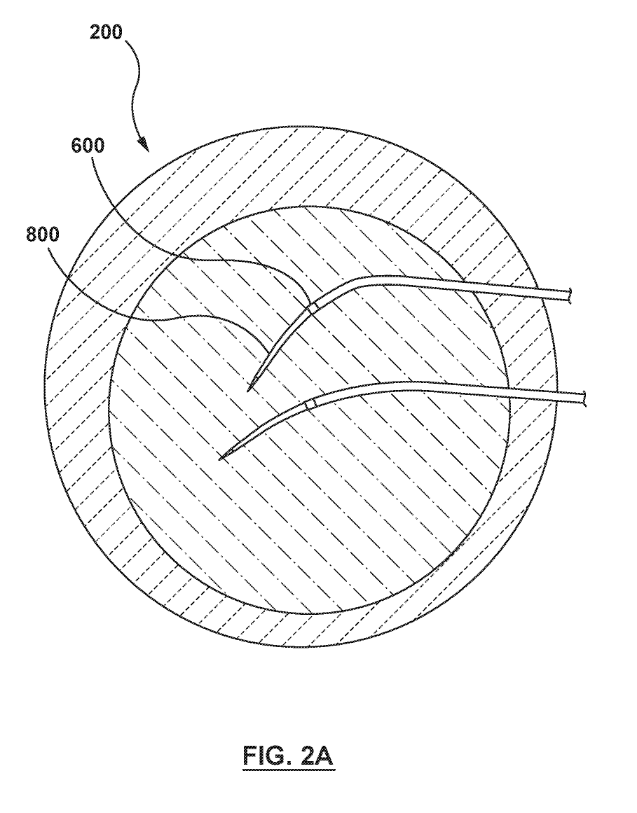

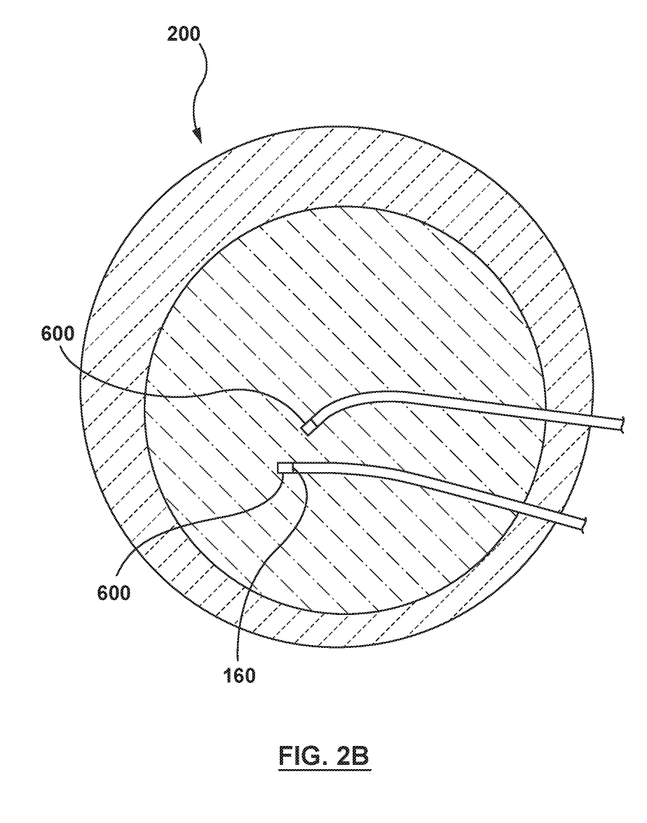

In accordance with some such examples of the present invention, a sheath assembly 200 is provided that provides enhanced radiopacity along the distal tip segment 600, as shown in FIGS. 2A-2B, showing a region of relatively sharp contrast distal of the marker band 160. FIG. 2A illustrates an embodiment of the invention under fluoroscopy. In this embodiment, a needle or dilator 700 is provided through a lumen of the sheath assembly 200. The distal tip segment 600 is markedly visible under fluoroscopy due to its enhanced radiopacity, allowing users to easily determine the distal end of the sheath assembly 200. In some embodiments, the sheath may be integral with the dilator and/or the needle. FIG. 2B illustrates the same embodiment as in FIG. 2A without a needle or dilator 800. Such, examples of the present invention, provide enhanced radiopacity while providing a distal tip wall thickness 600Tw that provides one or more advantages of: a substantially atraumatic tip and/or a smooth tip transition for a substantially smooth crossing.

In a specific example as additionally shown in FIGS. 1B and 1C, a section of 35D polymer substantially forms the distal tip segment 600, and is formed from an extrusion of an inner or under layer 604 of 35D polymer (40% Barium) having a length of about 1.5″ and an extrusion of an over layer or a second polymer layer 602 also of 35D polymer (40% Barium) has a length of about 0.5″, that are heat flowed to form the polymer layer 620 and in one example are both cut down to a length of about 0.18″. In one such example, the polymer layer 120 of the proximal shaft 110, such as Nylon 12 (25% barium) may be rolled over the under layer 604 of 35D polymer (that extends until marker band 160 of the proximal shaft 110). Then, over layer 602 of 35D polymer (40% Barium) is placed over both the under layer 604 of 35D polymer (40% Barium) and the polymer layer 120 of Nylon 12 (25% Barium) of the proximal shaft 110. The layers are then heat treated or heat flowed to form the lap joint 702, at the interface 700 of the distal tip segment 600 and the proximal shaft 110, just distal of the marker band 160, of the proximal shaft 110. As such, in some embodiments the distal tip segment 600 provides a region of enhanced radiopacity that allows the physician to see the distal tip profile that is distal of the marker band 160 under imaging. Even though the marker band 160 is dark and defines an area of sharp contrast that is easy to see, embodiments of the present invention allow for the distal tip segment 600 to be visible under imaging so its periphery or boundary is visible.

### Radiopacity of Distal Tip Section

In some embodiments, the radiopacity of the distal most section of the distal tip segment 600 (or section/region of increased wall thickness along the distal most section 600A) of the distal tip segment 600 has a radiopacity of between about 1.6 mmAl to about 1.9 mmAl. In some such examples, the distal most section 600A may be defined as the section of the distal tip segment 600 that is substantially distal of holes [h]. In one example, the radiopacity of the region of the region of increased wall thickness along the distal most section 600A of the distal tip segment 600 has a radiopacity of about 1.8 mm of Aluminum (mmAl). In one such example, the distal most section 600A has a length of about 0.105″ and the distal tip segment 600 has a length of about 0.18″, as additionally shown in FIG. 1A.

### Radiopacity of Proximal Section of Enhanced Wall Thickness

In some embodiments, the radiopacity of the section of enhanced wall thickness 600B of the distal tip segment 600 may have a radiopacity that is greater than a value of between about 1.6 mmAl to about 1.9 mmAl. In other words, the section of enhanced wall thickness 600B may have a radiopacity that is greater than the radiopacity of the distal most section (or distal tip section or section of the distal tip) 600A of the distal tip segment 600 due to the increased wall thickness in this area (which may be a gradual increase in radiopacity proximally as the wall thickness along the proximal section 600B increases proximally).

In other examples, the section of enhanced wall thickness 600B of the distal tip segment 600 may have a radiopacity of that is less than or equal to the radiopacity of the distal most section 600A of the distal tip segment 600 (which may be between about 1.6 mmAl to about 1.9 mmAl). In some such examples, one or more holes (for example two holes position at about 180 degrees (+/− about 30 degrees) from each other) are provided just distal to the proximal shaft for delivering contrast for instance, and may be positioned 0.105″ from the very distal edge of the distal tip segment 600. Another function of these holes is to facilitate aspiration and minimize cavitation. The removal of wall material from the section of enhanced wall thickness 600B (due to the holes [h]) may function to reduce the radiopacity in that segment. As such, distal most section 600A of the distal tip segment 600 may provide a region of enhanced radiopacity that is followed immediately proximally by an area lower radiopacity (compared to distal most section 600A) along the section of enhanced wall thickness 600B, as shown in FIGS. 2A and 2B. This may be followed immediately proximally by an area of sharp contrast or high radiopacity as defined by the marker band 10.

### Distal Tip Stiffness

In some embodiments, the stiffness of the distal most section 600A of the distal tip region 600 is less than about 0.8N, which in some embodiments may be defined as the force required to compress the tip by about 1 mm. In some embodiments, the stiffness of the distal most section 600A of the distal tip region 600 is less than about 0.5N. In a specific example, the sheath 200 has an average stiffness that is less than about 0.5N. In some embodiments, the stiffness of the distal most section 600A of the distal tip region 600 ranges from between about 0.25N to about 0.65 N. In some embodiments, the stiffness of distal most section 600A of the distal tip region 600 ranges from between about 0.4N to about 0.6 N.

### Tip Transition Force

In some embodiments of the present invention, the distal tip segment provides a smooth transition and requires a substantially less amount of force to cross the septum (i.e., less tip transition force). The tip transition force defines the force usable to advance the distal tip segment through a region of tissue (such as the septum for a transseptal procedure), where the sheath assembly is usable with a dilator and needle inserted therein (for example, an RF needle).

In some such embodiments of the present invention, the distal tip segment 600 has tip transition force of less than about 4N to cross a region of tissue. As still another feature of this broad aspect, the tip transition force is between about 0.5N to about 3.0N. In one specific example, the tip transition force is about 1.6N.

Tip Transition Force—Impact from Profile of the Distal Tip Section

In some embodiments of the present invention the distal tip segment 600 (including the distal most section [or distal tip section 600A] and the proximal section of enhanced wall thickness 600B) provide a smooth transition force. The distal tip segment 600 provides a wall thickness 600Tw in the distal most section 600A, that enhances radiopacity but provides a relatively low and/or smooth tip transition force (which in this case may be defined as the initial force to go through tissue). As such, in some embodiments of the present invention, distal tip segment 600 provides a region of increased wall thickness or thickened wall 606 along a portion/section of the distal tip segment 600 (or in other words at the tip) to enhance radiopacity while not compromising on crossing force, which in some examples, is the crossing force of the very distal tip (distal most section 600A).

In some examples of the present invention, the distal tip profile (or in other words, the outer diameter (OD) profile at the distal edge of the distal most section 600A) provides a gradual transition (for example over a dilator when in use with a dilator). In one configuration, a dilator (not shown) is positioned inside sheath assembly 200 such that the tip of the dilator protrudes from the end of the sheath. In this configuration, some such embodiments of the present invention provide a relatively small step in profile from the dilator to the sheath assembly 200. The distal profile of the distal most section 600A may help provide the physician with a smooth feel in puncture (for example using a needle positioned in the dilator) and smooth transition between the sheath and dilator as the sheath and dilator are advanced through the punctured tissue. As such, some embodiments of the present invention provide a thickened distal tip segment 600 having a region of increased wall thickness 606 where the tip thickness enhances radiopacity while reducing the tip transition force minimizing the force feel for the user to provide the user with a relatively smooth transition.

Tip Transition Force—Impact from Profile of the Proximal Section of Enhanced Wall Thickness

In some embodiments of the present invention, the tapered section or taper 608 of the proximal section of enhanced wall thickness 600B of the distal tip segment 600 helps to reduce the maximum amount of force a user may feel when advancing the distal tip segment 600 through a tissue wall, such as a septum. The taper 608 provides a gradual transition between the distal most section 600A and proximal section 600B and may help to minimize the change in force that is observed by the user as the distal tip is advanced through the tissue. This, in turn, may help reduce the risk that the distal tip section 600 deflects or jumps after a certain threshold of force is reached in order to cross the tissue. In other words, the taper 608 may help reduce/mitigate the risk of a jump of the distal tip segment 600 as it is being advanced.

### Tip Bond Force

Some embodiments of the present invention, as described herein above, provide a distal tip segment 600 that has a bond strength that is greater than about 15N. The bond strength may be defined as the force to disjoin or remove or dislocate the distal tip segment 600 from the proximal shaft 110. In some such embodiments, the distal tip segment 600 provides sufficiently high radiopacity while being soft enough to provide a substantially atraumatic tip and provide ease of crossability. As discussed above, various joints may be used between distal tip segment 600 and proximal shaft 110, such as lap joints or butt joints. The type of joint, materials used, dimensions of the surrounding structures, and the bonding agents used all have an impact on the tip bond strength.

Embodiments of the present invention as outlined herein above provide a modified tip with enhanced radiopacity while maintaining the distal tip profile to maintain adequate tip stiffness and tip transition force.

More specifically, embodiments of the present invention provide a distal tip segment 600 that provides a region of increased wall thickness along a portion thereof to provide enhanced radiopacity, while providing reduced profile along a distal most section of the tip, to help provide an atraumatic tip that is still sufficiently stiff to provide good transition force for crossability.

Some such embodiments of the present invention help provide greater or increased wall thickness along a portion of the distal tip segment to provide increased material density to provide enhanced radiopacity while providing adequate stiffness for atraumaticity and crossability. Some such embodiments may additionally provide good manufacturability and/or ease of manufacturability and/or consistency.

### Further Examples

1. A distal tip segment for a sheath assembly comprising:
   a region of increased wall thickness along a portion/section of the distal tip segment, providing increased cross-sectional area there-along to provide enhanced radiopacity; and
   a distal most/straight section (a region of reduced wall thickness) of the distal tip segment having a reduced/minimal profile to provide an atraumatic tip while maintaining sufficient stiffness for crossability.
   Wherein, the distal tip segment is operable to be coupled to a proximal shaft portion of the sheath assembly, to provide enhanced radiopacity while maintaining the distal tip profile to maintain adequate tip stiffness.
2. The distal tip segment of example 1, wherein the distal most section of the distal tip segment provides a reduced/good tip transition force to cross a region of tissue to facilitate a smooth transition.
3. The distal tip segment of example 1, wherein the distal tip segment additionally comprises a proximal section of enhanced thickness that defines the region of increased wall thickness along a proximal portion of the distal tip segment.
4. The distal tip segment of example 1, wherein the distal tip segment provides/comprises a gradual transition between the proximal section of enhanced wall thickness and the distal most section of the distal tip segment.
5. The distal tip segment of example 1, wherein the distal tip segment provides a gradual transition from the proximal shaft to the distal most section of the distal tip segment.
6. The distal tip segment of example 1, wherein the region of increased wall thickness along the distal most section of the distal tip segment has a wall thickness of about 0.010″ to about 0.014″.
7. The distal tip segment of example 6, wherein the region of increased wall thickness along the distal most section of the distal tip segment has a tip wall thickness of about 0.012″.
8. The distal tip segment of example 1, wherein the region of increased wall thickness along the proximal section of enhanced wall thickness has a maximum outer diameter that is less than about to about 0.158″.
9. The distal tip segment of example 1, wherein the distal tip segment has an inner diameter (along the region of increased wall thickness) that is in the range of between about 0.110 to about 0.112″.
10. The distal tip segment of example 9, wherein the distal tip segment has inner diameter along the region of increased wall thickness) that is equal to about 0.111″.
11. The distal tip segment of example 1, wherein the distal tip segment along the proximal section of enhanced wall thickness has a wall thickness that ranges from about 0.010″ at an interface with the distal most section of the distal tip region, to about 0.024″ at an interface with the proximal shaft portion.
12. The distal tip segment of example 5, wherein the distal tip segment along the region of increased wall thickness has a wall thickness that ranges from about 0.010″ at an interface with the region of reduced wall thickness to about 0.024″ at an interface with the proximal shaft portion.
13. The distal tip segment of example 1, wherein a lap joint is provided at an interface between the distal tip segment and the proximal shaft portion of the sheath assembly, wherein the lap joint defines the proximal of enhanced wall thickness.
14. The distal tip segment of example 11, wherein the lap joint is formed between a polymer layer of the proximal shaft portion and a polymer layer of the distal tip segment.
15. The distal tip segment of example 11, wherein the polymer layer of the proximal shaft portion is rolled down at the interface between the proximal section of enhanced wall thickness, of the distal tip segment, so it is tapered to enable the polymer layer of the distal tip segment to be joined/formed thereon in an overlapping configuration.
16. The distal tip segment of example 13, wherein the polymer layer of the proximal shaft portion comprises Nylon 12 and the polymer layer of the distal tip segment including the proximal section of enhanced wall thickness comprises a 35D Pebax.
17. The distal tip segment of example 14, wherein the polymer layer of the proximal shaft portion comprises 25% barium and the polymer layer of the region of increased wall thickness of the distal tip segment (including the distal most section of the distal tip region and the proximal section of enhanced wall thickness) comprises 40% barium to provide a region of enhanced radiopacity providing an area/region of sharp contrast.
18. The distal tip segment of example 15, wherein the polymer layer of the proximal shaft portion comprises Nylon 12 and the polymer layer of the distal tip segment comprises a 35D Pebax.
19. The distal tip segment of any one of examples 1 and 5, wherein the stiffness of the distal most section of the distal tip region is less than about 0.8 N.
20. The distal tip segment of any one of examples 1 and 5, wherein the stiffness of the distal most section of the distal tip region is less than about 0.5 N.
21. The distal tip segment of any one of examples 1 and 5, wherein the stiffness of the distal most section of the distal tip region ranges from between about 0.25 N to about 0.65 N.
22. The distal tip segment of any one of examples 1 and 5, wherein the stiffness of distal most section of the distal tip region ranges from between about 0.4 N to about 0.6 N.
23. The distal tip segment of any one of examples 1 and 5, wherein the radiopacity of the region of increased wall thickness along the distal most section of the distal tip segment has a radiopacity of between about 1.6 mmA to about 1.9 mmA.
24. The distal tip segment of example 22, wherein the radiopacity of the region of increased wall thickness along the distal most section of the distal tip has a radiopacity of about 1.8 (mm of Aluminum) mmAl.
25. The distal tip segment of any one of examples 1 and 10, wherein the radiopacity of the section of enhanced wall thickness of the distal tip segment has a radiopacity of that is greater than a value of between about 1.6 mmA to about 1.9 mmA.
26. The distal tip segment of example 1, wherein the distal tip segment has tip transition force of less than about 4N to cross a region of tissue.
27. The distal tip segment of example 1, wherein the tip transition force is between about 0.5N to about 3.0N.
28. The distal tip segment of example 23, wherein the tip transition force is about 1.6N.
29. The distal tip segment of any one of examples 26 to 28, wherein the tip transition force defines the force usable to cross the distal tip segment through a region of tissue, where the sheath assembly is usable with a dilator inserted therein/needle.
30. A sheath assembly comprising:
   a shaft portion comprising a proximal shaft portion,
   a distal tip segment distal of the distal end of the elongate member, the distal tip segment comprising:
      a proximal portion comprising a region of increased wall thickness, wherein the region of increased wall thickness has increased cross-sectional area for enhanced radiopacity; and
      a distal portion comprising a region of reduced wall thickness having a reduced cross-sectional area relative to the region of increased wall thickness to provide an atraumatic tip while maintaining sufficient stiffness for crossability;
   wherein the distal tip segment is operable to be coupled to the distal end of the shaft portion of the sheath assembly, to provide enhanced radiopacity while maintaining the distal tip profile to maintain adequate tip stiffness.

In one broad aspect, embodiments of the present invention comprise a distal tip segment for a sheath assembly comprising: a region of increased wall thickness along a portion/section of the distal tip segment, providing increased cross-sectional area there-along to provide enhanced radiopacity; and a distal most/straight section (a region of reduced wall thickness) of the distal tip segment having a reduced/minimal profile to provide an atraumatic tip while maintaining sufficient stiffness for crossability. The distal tip segment is operable to be coupled to a proximal shaft portion of the sheath assembly, to provide enhanced radiopacity while maintaining the distal tip profile to maintain adequate tip stiffness.

The embodiments of the invention described above are intended to be exemplary only. The scope of the invention is therefore intended to be limited solely by the scope of the appended examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the broad scope of the appended examples. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

We claim:

1. A distal tip segment for a sheath assembly comprising:
a region of increased wall thickness along a portion of the distal tip segment, providing increased cross-sectional area there-along to provide enhanced radiopacity; and
a distal most section which is straight comprising a region of reduced wall thickness of the distal tip segment having a reduced profile to provide an atraumatic tip while maintaining sufficient stiffness for crossability;
wherein the distal tip segment is operable to be coupled to a proximal shaft portion of the sheath assembly, to provide enhanced radiopacity while maintaining the distal tip profile to maintain adequate tip stiffness.

2. The distal tip segment of claim 1, wherein the distal most section of the distal tip segment provides a reduced tip transition force to cross a region of tissue to facilitate a smooth transition.

3. The distal tip segment of claim 1, wherein the distal tip segment additionally comprises a proximal section of enhanced wall thickness that defines the region of increased wall thickness along a proximal portion of the distal tip segment.

4. The distal tip segment of claim 3, wherein the distal tip segment comprises a gradual transition between the proximal section of enhanced wall thickness and the distal most section of the distal tip segment.

5. The distal tip segment of claim 1, wherein the distal tip segment provides a gradual transition from the proximal shaft to the distal most section of the distal tip segment.

6. The distal tip segment of claim 1, wherein the region of increased wall thickness along the distal most section of the distal tip segment has a wall thickness of about 0.010″ to about 0.014″.

7. The distal tip segment of claim 1, wherein the region of increased wall thickness along the distal most section of the distal tip segment has a tip wall thickness of about 0.012″.

8. The distal tip segment of claim 1, wherein the region of increased wall thickness along the proximal section of enhanced wall thickness has a maximum outer diameter that is less than about to about 0.158″.

9. The distal tip segment of claim 1, wherein the distal tip segment has an inner diameter along the region of increased wall thickness that is in the range of between about 0.110 to about 0.112″.

10. The distal tip segment of claim 1, wherein the distal tip segment has an inner diameter along the region of increased wall thickness that is equal to about 0.111″.

11. The distal tip segment of claim 3, wherein the distal tip segment along the proximal section of enhanced wall thickness has a wall thickness that ranges from about 0.010″ at an interface with the distal most section of the distal tip region, to about 0.024″ at an interface with the proximal shaft portion.

12. The distal tip segment of claim 5, wherein the distal tip segment along the region of increased wall thickness has a wall thickness that ranges from about 0.010″ at an interface with the region of reduced wall thickness to about 0.024″ at an interface with the proximal shaft portion.

13. The distal tip segment of claim 3, further comprising a lap joint at an interface between the distal tip segment and the proximal shaft portion of the sheath assembly, wherein the lap joint defines the proximal section of enhanced wall thickness.

14. The distal tip segment of claim 13, wherein the lap joint is formed between a polymer layer of the proximal shaft portion and a polymer layer of the distal tip segment.

15. The distal tip segment of claim 14, wherein the polymer layer of the proximal shaft portion is rolled down at the interface between the proximal section of enhanced wall thickness, of the distal tip segment, so it is tapered to enable the polymer layer of the distal tip segment to be joined/formed thereon in an overlapping configuration.

16. The distal tip segment of claim 14, wherein the polymer layer of the proximal shaft portion comprises Nylon 12 and the polymer layer of the distal tip segment including the proximal section of enhanced wall thickness comprises a 35D Pebax.

17. The distal tip segment of claim 14, wherein the polymer layer of the proximal shaft portion comprises 25% barium and the polymer layer of the region of increased wall thickness of the distal tip segment which includes the distal most section of the distal tip region and the proximal section of enhanced wall thickness comprises 40% barium to provide a region of enhanced radiopacity providing an area of sharp contrast.

18. The distal tip segment of claim 15, wherein the polymer layer of the proximal shaft portion comprises Nylon 12 and the polymer layer of the distal tip segment comprises a 35D Pebax.

19. The distal tip segment of claim 1, wherein the stiffness of the distal most section of the distal tip region is less than about 0.8 N.

20. The distal tip segment of claim 1, wherein the stiffness of the distal most section of the distal tip region is less than about 0.5 N.

21. The distal tip segment of claim 1, wherein the stiffness of the distal most section of the distal tip region ranges from between about 0.25 N to about 0.65 N.

22. The distal tip segment of claim 1, wherein the stiffness of distal most section of the distal tip region ranges from between about 0.4 N to about 0.6 N.

23. The distal tip segment of claim 1, wherein the radiopacity of the region of increased wall thickness along the distal most section of the distal tip segment has a radiopacity of between about 1.6 mmAl to about 1.9 mmAl.

24. The distal tip segment of claim 1, wherein the radiopacity of the region of increased wall thickness along the distal most section of the distal tip has a radiopacity of about 1.8 mmAl.

25. The distal tip segment of claim 3, wherein the radiopacity of the proximal section of enhanced wall thickness of the distal tip segment has a radiopacity of that is greater than a value of between about 1.6 mmAl to about 1.9 mmAl.

26. The distal tip segment of claim 1, wherein the distal tip has a tip transition force which is defined as the force usable to cross the distal tip segment through a region of tissue, where the sheath assembly is usable with a dilator and/or needle inserted therein.

27. The distal tip segment of claim 26, wherein the tip transition force is less than about 4N to cross a region of tissue.

28. The distal tip segment of claim 26, wherein the tip transition force is between about 0.5N to about 3.0N to cross a region of tissue.

29. The distal tip segment of claim 26, wherein the tip transition force is about 1.6N to cross a region of tissue.

30. A sheath assembly comprising:
a shaft portion comprising a proximal shaft portion,
a distal tip segment distal of the distal end of the elongate member, the distal tip segment comprising:
a proximal portion comprising a region of increased wall thickness, wherein the region of increased wall thickness has increased cross-sectional area for enhanced radiopacity; and
a distal portion comprising a region of reduced wall thickness having a reduced cross-sectional area relative to the region of increased wall thickness to provide an atraumatic tip while maintaining sufficient stiffness for crossability;
wherein the distal tip segment is operable to be coupled to the distal end of the shaft portion of the sheath assembly, to provide enhanced radiopacity while maintaining the distal tip profile to maintain adequate tip stiffness.
